(19) United States
(12) Patent Application Publication
(10) Pub. No.: US 2026/0345104 A1
(43) Pub. Date: Nov. 5, 2026

(54) SYSTEM AND METHOD FOR DETERMINING GEOMETRY OF ANATOMICAL FEATURES FROM MEDICAL IMAGES

(71) Applicant: Medtronic, Inc., Minneapolis, MN (US)

(72) Inventors: Nikhil MAHENDRA, Broomfield, CO (US); Abhijeet A. DUBHASHI, Cotati, CA (US); Jacob SCHNEIDER-MARTIN, San Francisco, CA (US); Madeline M. TUNENDER, Sioux Falls, SD (US); Srinivasan S. VARAHOOR, Windsor, CA (US); Yvan R. PAITEL, Louisville, CO (US); Kaustubh R. PATIL, Plymouth, MN (US); Jeannine ELLIOTT, Superior, CO (US)

(21) Appl. No.: 19/485,926

(22) PCT Filed: Jun. 25, 2024
(86) PCT No.: PCT/IB2024/056161

§ 371 Date: Nov. 19, 2025

Related U.S. Application Data
(60) Provisional application No. 63/523,402, filed on Jun. 27, 2023.

(51) Int. Cl. A61B 6/50; A61B 6/00; A61B 6/03; A61B 34/10; A61F 2/24; G06T 7/00; G06T 7/11; G06T 7/62

(52) U.S. Cl. CPC A61B 6/503; A61B 6/032; A61B 6/5217; A61B 34/10; A61F 2/24; G06T 7/0012; G06T 7/11; G06T 7/62; A61B 2034/108; G06T 2207/10081; G06T 2207/20081; G06T 2207/20084; G06T 2207/30048

(57) ABSTRACT

A three-dimensional (3D) image of a cardiac region is input to a convolutional neural network (CNN) to semantically segment the 3D image. An output of the CNN includes a 3D geometry of a heart valve structure within the cardiac region. A chain code is applied to the 3D geometry to obtain two-dimensional (2D) geometric parameters of the heart valve structure. The 2D geometric parameters are used to recommend or select an implantable device that fits with the heart valve structure.

### CROSS-REFERENCE TO RELATED APPLICATION

This application claims the benefit of U.S. Provisional Patent Application Ser. No. 63/523,402, filed Jun. 27, 2023, the entire content of which is incorporated herein by reference.

### FIELD

The present technology is generally related to machine-learning-based analysis of three-dimensional medical images.

### BACKGROUND

Medical imaging is an instrumental tool in medical diagnosis and treatment. Three-dimensional (3D) medical imaging has gained widespread adoption in the medical field. Three-dimensional medical imaging provides a detailed and accurate visualization of internal structures and tissues, which can be used to diagnose and treat complex medical conditions.

Three-dimensional medical imaging is achieved through a variety of imaging techniques such as computed tomography (CT), magnetic resonance imaging (MRI), and ultrasound. These techniques create three-dimensional images of organs, bones, tissues, and other structures in the body, providing practitioners with a detailed and accurate representation of the patient's internal anatomy. With the ability to view the body from multiple angles, medical professionals can make more precise diagnoses, plan complex surgical procedures, and improve patient outcomes.

It can sometimes be difficult to find features (e.g., anatomical structures) in a 3D image. The image data is typically stored in a 3D array which may be viewed on as a 3D image on a 2D display, and the structures of interest can sometimes be obscured by other anatomical features making them hard to locate in in 3D renderings, or even in smaller views (e.g., sub-arrays of the image structure). Viewing the image as two-dimensional (2D) sections can bypass obscuring structures. However, because anatomical features are geometrically complex organic shapes that can differ significantly between individuals and will typically not be aligned with viewing planes, they can sometimes be hard to identify in 2D views. Because of the complexities involved in presenting and manipulating 3D images, some medical 3D imaging tasks can be time consuming to perform, resulting in significant costs being associated with those tasks.

### SUMMARY

The present disclosure is directed to determining geometry of anatomical features from medical imaging. In one embodiment, a method involves obtaining a three-dimensional (3D) image of a cardiac region. The 3D image is input to a convolutional neural network (CNN) to semantically segment the 3D image. An output of the CNN includes a 3D geometry of a heart valve structure within the cardiac region. A chain code is applied to the 3D geometry to obtain two-dimensional (2D) geometric parameters of the heart valve structure. The 2D geometric parameters are used to recommend or select an implantable device that fits with the heart valve structure.

In one embodiment, the heart valve structure includes a mitral valve annulus, and the implantable device comprises a transcatheter mitral valve replacement system. In such a case, the 2D geometric parameters may include the following parameters of the mitral valve annulus: an inter-commissural diameter, an anterior-posterior diameter, a perimeter, and an area. In one embodiment, inputting the 3D image into the CNN involves: inputting a 3D heart scan image into a first CNN that produces a bounding volume that contains the heart valve structure; bounding the 3D heart scan image by the bounding volume; and inputting the bounded image to a second CNN to obtain the 3D geometry of the heart valve structure. In another embodiment, the method further involves training the CNN on patient data sets in which a clinician manually defines a 2D outline on a 3D heart image. The 2D outline defines at least one boundary of an equivalent heart valve structure. In some embodiments, the 3D geometry of the heart valve structure has a volume that is less than 0.1% of the 3D image. Applying the chain code to the 3D geometry may involve projecting the 3D geometry to a plane, the chain code being applied in 2D to the projection. The 3D image may include a 3D video image.

These and other features and aspects of various embodiments may be understood in view of the following detailed discussion and accompanying drawings.

### BRIEF DESCRIPTION OF THE DRAWINGS

The discussion below makes reference to the following figures, wherein the same reference number may be used to identify the similar/same component in multiple figures.

FIG. 1 is a diagram showing a heart structure analyzed using systems and methods according to an example embodiment;

FIG. 2 is a medical image overlaid with geometric shapes which can be used for training a neural network according to example embodiments;

FIG. 3 is a diagram showing a set of convolutional neural networks according to example embodiments;

FIG. 4 is a set of medical images showing the location of a target anatomical structure within a 3D image file according to an example embodiment;

FIG. 5 is a diagram showing details of convolutional neural networks according to example embodiments;

FIG. 6 shows an example of processing performed on a neural network identified 3D structure according to an example embodiment;

FIGS. 7 and 8 are diagrams showing implementation of a chain code according to an example embodiment;

FIG. 9 is a diagram showing a projection of a 3D shape to a 2D plane according to an example embodiment;

FIG. 10 is a block diagram of a system and apparatus according to an example embodiment; and

FIG. 11 is a flowchart of a method according to an example embodiment.

### DETAILED DESCRIPTION

The present disclosure is generally related to systems and methods for segmenting and measuring anatomical features in a 3D medical image using a machine-learning model. For purposes of illustration, these embodiments are described in the context of identifying and measuring heart structures, such as mitral valve openings. It will be understood that the systems and methods may be used for other types of anatomical identification and measurement, both in the heart and other parts of human and animal anatomy.

Mitral valve regurgitation is a type of heart valve disease in which the valve between the left heart chambers doesn't close completely, allowing blood to leak backward across the valve. Patients with symptomatic primary or secondary mitral regurgitation (MR) may be considered for transcatheter mitral valve repair, which involves introducing a clip into a heart chamber through a catheter. The clip holds edges of the valve together in the middle, reducing or preventing the backflow of blood.

Another MR treatment is known as transcatheter mitral valve replacement, in which a prosthesis is placed within the annulus of the mitral valve. The prosthesis is a self-expanding stent that is compressed inside a hollow delivery catheter through which it is delivered to the heart. Once in place, an outer part of the stent conforms to the native annulus without need for additional sutures, tethers, or anchors. A bovine pericardium valve can be housed within an inner part of the stent to replace the function of the failed mitral valve.

Patients eligible for the valve replacement undergo contrast-enhanced, cardiac computed tomography (CT) of the mitral valve annulus to determine suitability and choice of the prosthesis size (42mm, 48 mm, etc.). In general, a prosthesis size is chosen that will allow 10% to 30% oversizing in the mitral annular perimeter, inter-commissural (CC) diameter and anterior-posterior (AP) diameter, while minimizing risk of left ventricular outflow tract (LVOT) obstruction. A machine-learning-based model may be implemented to identify the aortic plane in the CT image. Once the aortic plane is identified, a center line is traced from the aortic annulus to the left ventricular outflow tract (LVOT). This center line serves as a reference point for placing a virtual cone parallel to the edge to mark the neo-LVOT. By using this marker and virtual cone, the neo-LVOT area can be calculated along the entire virtual valve to determine the optimal device size.

The pre-screening and planning for each case is currently done manually by clinicians. The current screening time is about six hours per patient performed by a patient screening specialist. This disclosure describes a method to automatically detect and measure the mitral valve annulus. This can improve efficiency of the procedure planning phase of implanting catheter-based valves, e.g., by reducing an amount of time needed by a specialist in analyzing and annotating anatomical structures in 3D imagery. The methods described herein could be extended for annulus detection in tricuspid (TTVR) and detecting three points of the basal plane for aortic valve (TAVR) planning.

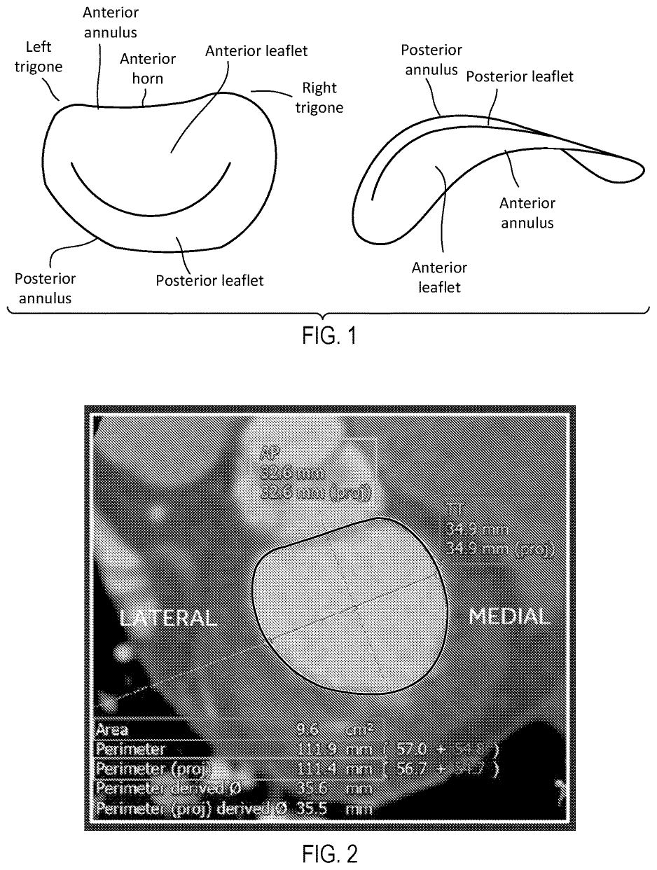

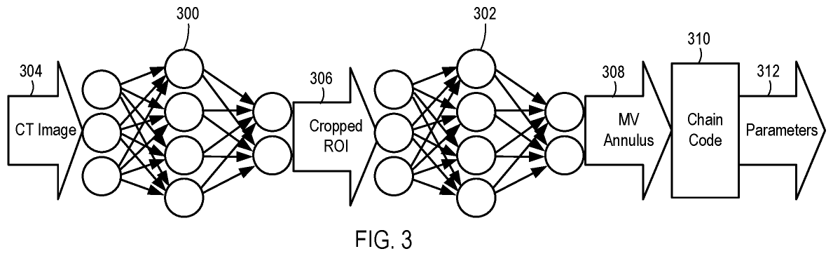

The mitral annulus has a nonplanar, saddle-shaped, 3D structure as shown in the simplified two-dimensional (2D) view (left side) and 3D view (right side) of FIG. 1. The mitral annulus includes the anterior and posterior annuli shown in FIG. 1. In FIG. 2, a screenshot of an imaging program shows a manually identified mitral valve annulus using a CT image. With the anterior horn excluded, a planar “D-shaped” annulus with its anterior border is defined by a virtual line connecting both trigones. This abstracted/ simplified annulus shape is utilized for sizing. After defining the annular shape, geometric parameters can be derived from the shape, including the AP diameter (AP proj), CC diameter (inter-trigonal (TT) proj), mitral valve area (area) and 2D perimeter (perimeter proj).

The D-shaped annulus structure, which slightly changes in size and geometry during the cardiac cycle, is a challenging structure to detect and measure automatically. With the advent of transcatheter mitral valve replacement (TMVR), the automatic assessment of mitral annular dimensions is of increasing relevance.

In embodiments described below, machine learning models are used to automatically detect anatomic structures in 3D images. It will be understood that the term “3D images” as used herein refers to three spatial dimensions, and the machine learning models may take as input (and be trained on) 3D video images, which include a time series of 3D still images, and may be stored using video compression algorithms. In this case, the time may be considered a fourth dimension for purposes of constructing machine learning models. The specific examples described below pertain to detecting and defining geometry of the mitral valve annulus in CT images, however these techniques are applicable to other imaging modalities, including but not limited to magnetic resonance imaging (MRI), ultrasound, positron emission tomography (PET), X-ray, etc.

In other embodiments of the methods described herein, the machine learning models are used to automatically detect anatomic structures in 3D images. It will be understood that the term “3D images” as used herein refers to three spatial dimensions, and the machine learning models may take as input (and be trained on) 3D video images, which include a Weighted Temporal Average or Weighted Temporal Variance image constructed from the entire time series of 3D images. The images may be stored using video compression algorithms.

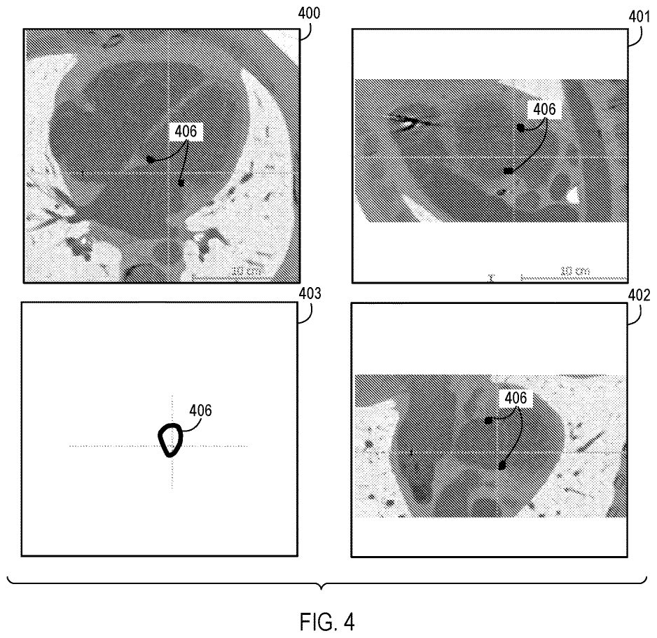

As shown in the block diagram of FIG. 3, a deep learning-based architecture in one or more embodiments uses two neural networks 300, 302. The neural networks include an initial localization network 300 that takes full CT image 304 as input and outputs region of interest (ROI) 306, e.g., a mask with a bounding box around the mitral valve. This smaller cropped ROI 306 is given as input to the second neural network 302, which performs segmentation to determine the shape of mitral valve annulus structure 308. The detected mitral annulus structure 308 is then processed via chain code block 310 to calculate geometric parameters 312 such as perimeter, area, AP and CC diameters. The geometric parameters 312 may be based on a 2D projection of the 3D shape of the identified valve structure 308. The chain code block 310 utilizes a chain code (e.g., Freeman chain code) to represent the structure and derive the parameters 312. Other steps may be performed in this process that are not shown, such as finding a largest connected component, noise-removal, smoothing, etc.

The networks 300, 302 may use a number of machine-learning algorithms. The machine-learning algorithm(s) may include one or more of a convolutional neural network (CNN) algorithm, an autoencoder algorithm, a recurrent neural network (RNN) algorithm, and transformer neural network algorithm, a generative adversarial network (GAN) algorithm, linear regression, support vector machine (SVM) algorithm, random forest algorithm, hidden Markov model, and/or any combination thereof. For example, in some embodiments, the at least one processor may be configured to utilize a combination of a CNN algorithm in conjunction with an SVM algorithm.

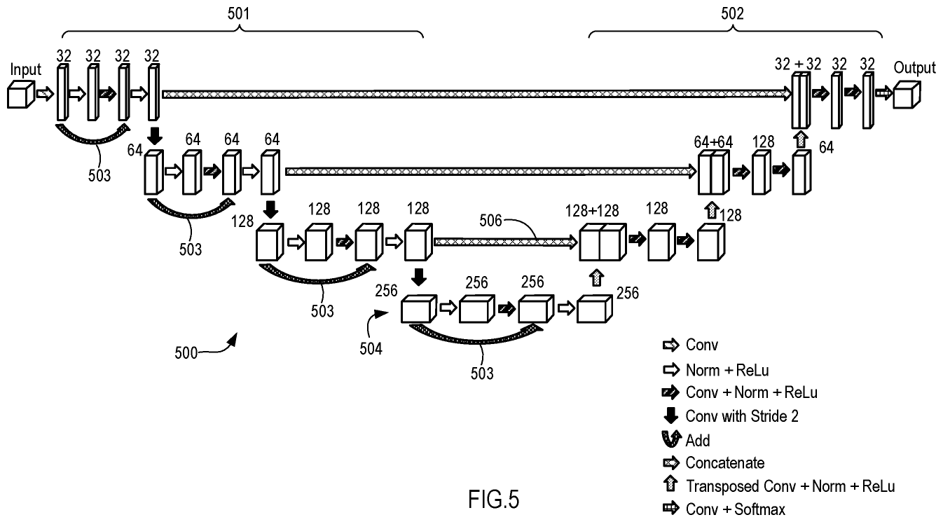

Before describing details of the architecture shown in FIG. 3, the images in FIG. 4 are used to demonstrate the input domain that informed the design of the architecture according to various embodiments. Images 400-402 are views of a mitral annulus 406 from various angles including the surrounding tissue. Image 403 shows the isolated geometry of the annulus 406. In this case, the image sizes are 512×512×173, which comes to 45,350,912 voxels. A bounding volume (also referred to herein as a bounding box) around the annulus 406 is around 13,000 voxels, therefore has a volume that is around 0.029% of the total volume of the 3D image. Thus, by using the localization neural network 300 shown in FIG. 3 to isolate the annulus geometry, the segmentation neural network 302 can produce more accurate results as it is trained on a smaller region. Generally, a localization neural network 300 may be beneficial for a target structure volume that is less than n % of the 3D image volume, wherein n =1%, 0.1%, 0.05%, etc.

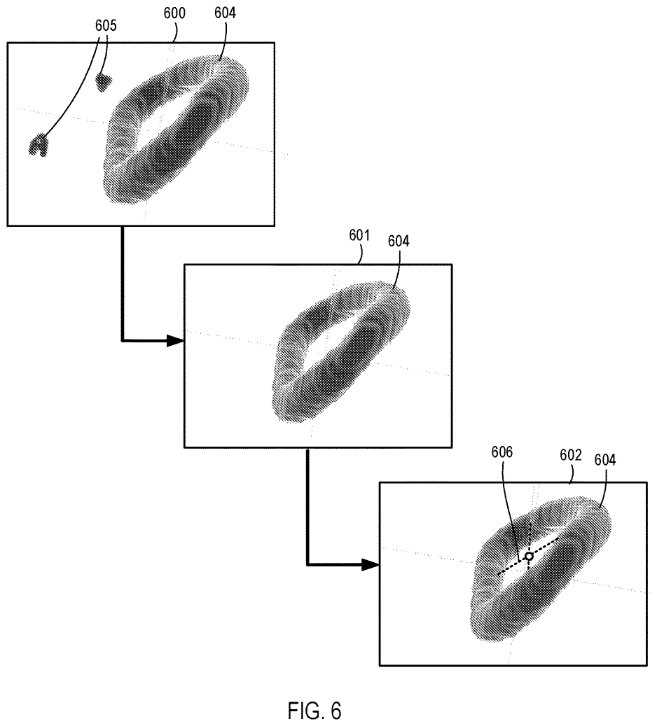

In FIG. 5, a block diagram illustrates a neural network architecture 500 according to an example embodiment. This type of neural network architecture 500 can be used for both the localization and segmentation networks 300, 302 shown in FIG. 3, with appropriate adjustments for input image size and other changes as described below. In other embodiments, the localization and segmentation networks 300, 302 may have different architectures entirely, e.g., selected from the architectures described in relation to FIG. 3. Because the bounding box that defines the identified annulus (e.g., annulus 406 in FIG. 4) may vary for different patients, the cropped image may be resampled to fit an input size of the segmentation network 302. In other embodiments, the bounding box may be padded with zeros or the like to fill out the input layer of the segmentation network 302. In other cases, different sized bounding boxes may be used as input to network 302. For example, different versions of the segmentation network 302 may be trained for different sized bounding boxes.

The network architecture 500 in FIG. 5 is based on the 3D U-Net and Link-Net architectures. The U-Net architecture is described in “3D U-Net: Learning Dense Volumetric Segmentation from Sparse Annotation” by Özgün Çiȩek et al. (arxiv.org/pdf/ 1606.06650.pdf) and is a type of convolutional neural network (CNN). Link-Net is described in Abhishek Chaurasia et al., “LinkNet: Exploiting Encoder Representations for Efficient Semantic Segmentation,” (arxiv.org/pdf/1707.03718.pdf).

The left side of the network is an analysis path 501 (also referred to as a contracting path or encoder part) and the right side is a synthesis path 502 (also referred to as an expanding path or decoder part). The boxes in each layer represent feature maps, and the number of channels is denoted above each feature map. Note that the channels shown in this example embodiment are for the localization network 300. For a corresponding embodiment of the segmentation network 302, the number of channels for layer 504 is 128, and the number of channels involved in the concatenation 506 is 64+128.

The network 500 uses the basic architecture of U-Net network with a contracting encoder part 501 to analyze the image and a successive expanding decoder part 502 to synthesize and produce a full-resolution segmentation mask. The encoder blocks have additional residual connections 503 not used in the U-Net paper. The additional residual connection 503 are for feature extraction and are similar to convolution modules defined in the LinkNet paper. Each convolution module in this network 500 contains batch normalization and rectified linear unit (ReLu) activation layers, which differs from both LinkNet and U-Net. The number of filters is different from the defaults defined in UNet paper. Strided-convolution layers are used instead of max-pooling for feature downsampling. See, e. g, Ayachi, et al. (2020); Strided Convolution Instead of Max Pooling for Memory Efficiency of Convolutional Neural Networks; Proceedings of the 8th International Conference on Sciences of Electronics, Technologies of Information and Telecommunications (SETIT'18); doi.org/10.1007/978-3-030-21005-2_23.

In the analysis/encoder path 501, each layer contains two (3×3×3) convolutions each followed by batch normalization and a ReLu activation, and then a (3×3×3) strided convolution with strides of two in each dimension. A dropout layer is added after the second convolution of each layer before ReLu activation to avoid overfitting. In the synthesis/decoder path 502, each layer includes a transposed convolution of (3×3×3) by strides of two in each dimension, followed by batch normalization and ReLu activation, following by two (3×3×3) convolutions each followed by batch normalization and ReLu. Shortcut connections (curved arrows) from layers of equal resolution in the analysis path provide the targeted high-resolution features to the synthesis path 502.

The datasets utilized for training the networks are from TMVR device trials and images from the internally generated datasets. In these datasets, comparable 3D images (e.g., CT images) were manually annotated as shown in FIG. 2. Images from datasets are resampled to fixed size isotropic voxel sizes and their intensities normalized by scaling between 0 to 1 before passing as input to the neural network. These preprocessing steps help the network see inputs with more uniform sizes and intensities across the dataset and would also be performed on operational images input to the networks during use of the system. The training dataset is also augmented with random rotations, translations, intensity scaling, variation and adding noise to the images. This can help improve the network performance with a smaller training set.

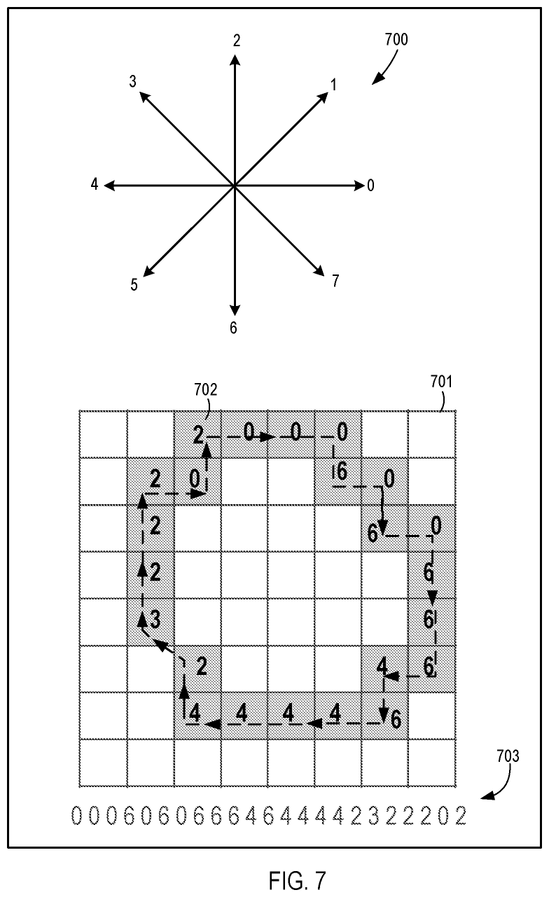

In FIG. 6, a series of images shows additional processing that may be performed on a detected anatomical feature according to an example embodiment. As seen in image 600, a mitral valve annulus shape 604 has been identified via neural networks as shown in FIG. 3, along with unwanted artifacts 605. As seen in image 601, the artifacts have been removed e.g., by identifying the largest connected component (the annulus shape 604) or other noise reduction technique. As seen in image 602, the chain code processing has been used to identify geometric parameters 606 as described above, e.g., diameters, area, centroid, etc.

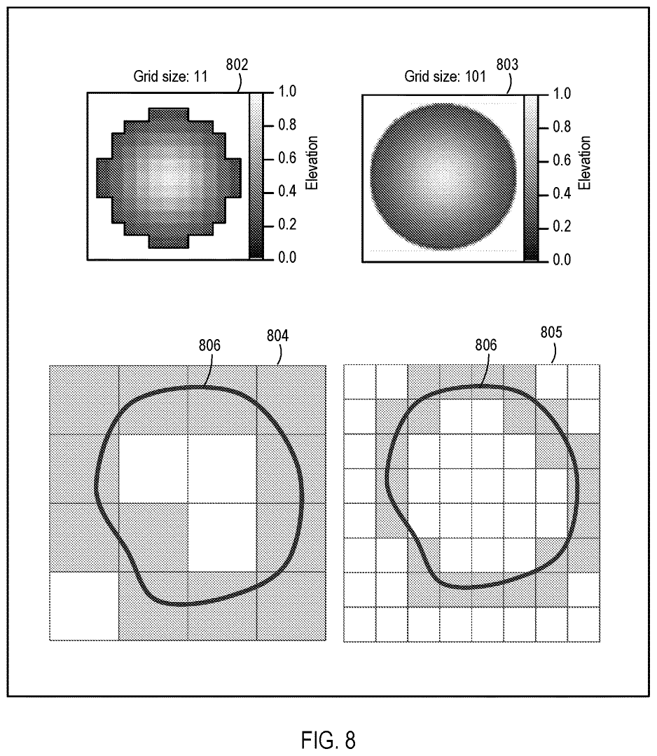

In FIG. 7, a diagram shows an example of concepts related to chain codes used in systems and methods according to example embodiments. Diagram 700 is an example of different 2D directions that are mapped to codes. The codes are single digits from 0-7 (e.g., may use an octal numbering system) in this example and the directions are four directions normal to the sides of a square and four diagonal directions corresponding to the corners of the square. This implementation is referred to as a Freeman code, and other chain codes can be used, e.g., using 2D a map with no diagonals, etc. The diagram 700 in FIG. 7 shows a 2D code, however this can be extended to 3D, e.g., by using a hexadecimal code mapped to 14 directions, six directions normal to the sides of a cube and eight diagonal directions.

The grid 701 in FIG. 7 shows how the code is used to encode a shape, which is indicated by the shaded squares in the grid 701. A blob or kernel is defined that detects the center of regions with similar properties, e.g., greyscale intensity, which can then be used as a binary indicator of inclusion in the shape. A start location 702 is determined, and for each adjacent square, the code 703 is formed by appending the digit corresponding to the direction from which the square was entered. As shown, the encoding begins at location 702 and the first digit of the code 703 is zero, which corresponds to the first transition from the start location 702 to the next square to the right. Digits are appended to the code 703 in this way as the shape is traversed, indicated by the dashed line through the grid 701. Because this example is a closed shape, the code 703 ends when the start location 702 is entered.

In FIG. 8, a diagram shows additional details of a processing geometry according to an example embodiment. This figure illustrates how a continuous real-world structure, such as the closed shape 806, is represented in a discretized format when digitized as a 2D image with a pixelized representation of the line shown in grey in grids 804 and 805. The pixel (voxel) grid sizes are determinative in the ability to represent the anatomical structures with higher accuracy. In one embodiment, the input CT images are resampled to a higher resolution grid (voxel) size before inputting them to the segmentation network 302 for this reason.

In rasterization, a continuous vector image is converted into a grid of pixels. The accuracy of the resulting raster image depends on the resolution of the grid, which is determined by the number of pixels per unit of length. If the grid size is too large, the resulting image may lose critical shape information and appear pixelated or jagged. On the other hand, a higher resolution (e.g., a smaller grid size with more pixels as seen in grid 804) can provide a more accurate representation of the original vector image.

Therefore, the use of a high resolution or smaller grid size (as shown in grid 805) with more pixels in rasterization is similar to the use of a high resolution or smaller grid size in digitizing a 2D image of a continuous real-world structure in order to accurately represent the structure and avoid losing relevant information.

Even if the segmentation was 100% accurate, but the voxel sizes were too large, that could still lead to inaccurate calculation of the measurements such as perimeter, area, diameter because of the lower-resolution representation of the valve. Plots 802 and 803 show how the shape of a circle changes with respect to the grid size. A grid size of 11 has too low resolution and would miss critical features of the shape of a circle, compared to a higher resolution image with grid size of 101 pixels showing circle shape more clearly.

In the embodiments described above, the chain code is applied to a segmented structure or shape that is output from the neural networks. The result of the chain code is an abstract geometry (e.g., connected line segments, connected curve) that estimates geometric features which can be parameterized. For example, a centroid of the shape can be estimated by determining a centroid of the greyed squares. Calculations for other geometric parameters shown in FIG. 2 can be obtained in a similar way, e.g., estimating the shape as a piecewise linear shape where each line segment extends between the centers of adjacent squares in the shape. The same calculations can be performed for different blob/kernel sizes to see if one or more calculated parameters agree (or conform to an expected trend) over different scales.

The chain codes described above can be performed on a 2D projection of the segmented 3D shape that is output from the neural networks. In other embodiments, a 3D chain code can be used, e.g., where plots such as 802 and 803 correspond to spheres instead of circles, and the grids 804, 805 correspond to volumes. The parameterized shape estimate can then be used to find an appropriate plane in which to project the shape, after which 2D geometric parameters can be derived.

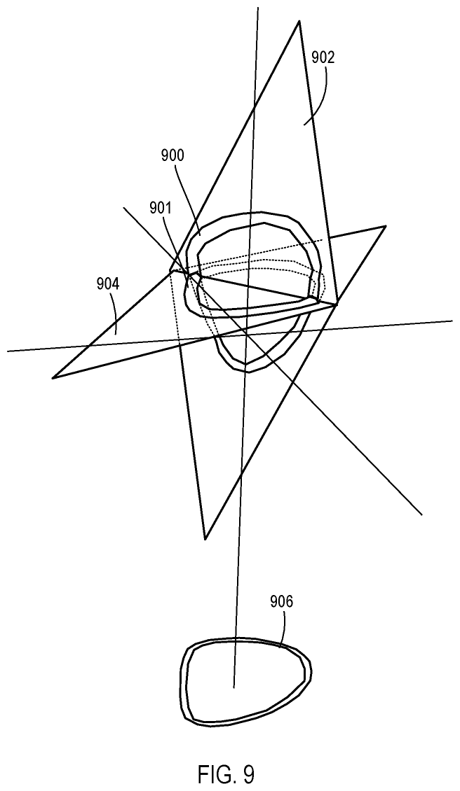

In FIG. 9, a block diagram shows an example of projecting a 3D geometry to a 2D geometry according to an example embodiment. In this example, an anatomical geometry (e.g., mitral annulus 900) is shown as it is segmented as previously described. Plane 902 represents a native axis of the valve. Plane 904 is the orthogonal axis of image planes (axial, sagittal, coronal) and is rotated relative to the plane 902 such that a projection 906 of the annulus 901 after the rotation accurately captures the relevant geometry, e.g., maximizes the AP and CC diameters of the projection 906. The plane 904 may be found, for example, by a least squares regression which comprises a best fit plane for all of the detected mitral valve points. The projection 906 can be processed using a 2D chain code as described above to determine the geometric parameters. Alternatively, a 3D chain code can be applied to determine a 3D path of the annulus 900, and this 3D path can be projected onto plane 904, the plane being determined based on the chain code path instead of the 3-D structure of the annulus 900. The final outcomes of either process are the geometric parameters, e.g., AP and CC diameters, area, etc., as described above.

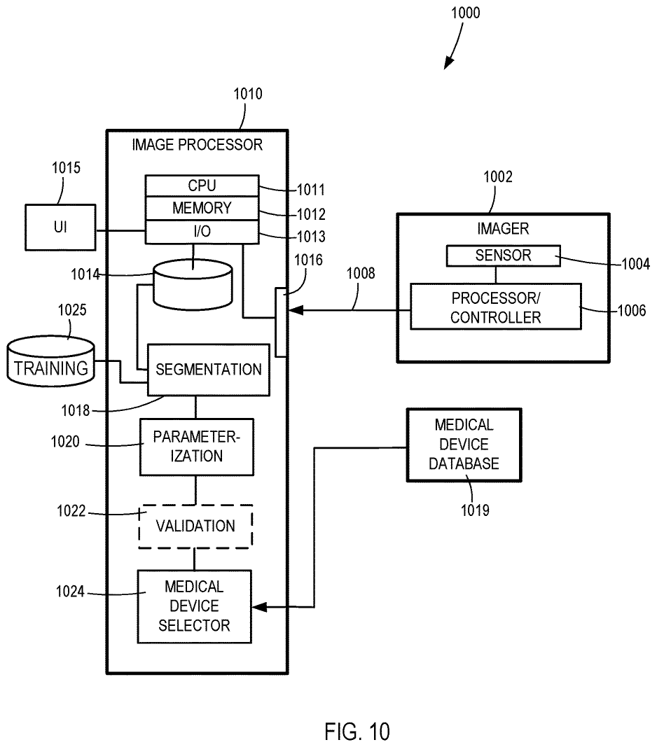

In FIG. 10, a block diagram shows a medical imaging system 1000 according to an example embodiment. The system includes an imaging device 1002 such as a CT scanner, MRI scanner, ultrasound scanner, etc. The imaging device 1002 includes one or more sensors 1004 that can detect anatomical features via emitted and/or reflected energy, e.g., electromagnetic waves, sound etc. The imaging device 1002 may include processing and control circuitry 1006 that receives, buffers, processes and outputs images 1008 to an image processor 1010. The processing performed by the processor/controller may include low level digital signal processing (e.g., analog-to-digital conversion, filtering, phase shifting, etc.) such that the images 1008 are in a format recognizable by the image processor 1010, e.g., industry standard digital image and video file formats.

The image processor 1010 may include conventional computing hardware such as a central processing unit (CPU) 1011, memory 1012, input/output (I/O) interfaces 1013, and a non-volatile data storage unit 1014 (e.g., hard disk drives, solid state drives). A user interface 1015 is also coupled to or included with the image processor 1010, e.g., monitor, keyboard, mouse, trackball, virtual reality or augmented reality viewer, etc.

The image processor 1010 includes an external data interface 1016 that receives the images 1008 from the imaging device 1002. The data storage unit 1014 may locally store the images for further processing. The image processor 1010 may be a separate computing device (e.g., general purpose computer) or may be integrated with the imaging device 1002 and/or user interface 1015 in a single physical unit.

The images 1008 are processed via a segmentation unit 1018 which uses one or more CNNs to identify a region of interest and well as identify particular anatomical structures such as a mitral valve annulus. The identified structure is passed to a parametrization unit 1020 which simplifies the identified structure, e.g., projecting to a 2D view, fitting a simplified shape to the structure using a chain code, etc. This provides a set of geometric parameters as described above that can be used for fitting an implantable device. An optional validation unit 1022 can provide the machine-learning derived results to a user to validate via the user interface 1015, e.g., overlay the derived structures and shapes on a 3D view of the original images.

A medical device selector unit 1024 can match the geometric parameters obtained from the parameterization unit 1020 with available medical devices, e.g., by accessing a local or remote medical device database 1019. The medical device selector unit 1024 may use a lookup table or the like to match an available device or class of devices to the particular anatomic geometry that was identified. For example, the mitral valve annulus prosthesis may come in multiple sizes (e.g., 42 mm, 48 mm, etc.) and the individual geometric parameters (AP diameter, CC diameter) and/or combinations of the parameters (e.g., ratio of AP diameter to CC diameter) may be applied to via an algorithm (e.g., lookup table, rules-based system, heuristics) to determine suitability and choice of the prosthesis size.

The segmentation unit 1018 may use pretrained neural networks. Generally, the training may occur on a different system than the image processor 1010, however the training arrangement may have analogous hardware to the image processor 1010, e.g., CPU, memory, etc. The training utilizes training data 1025 that may include patient data sets in which a clinician manually defines a 2D or 3D outline on a 2D or 3D heart image. For example, the 2D outline may be a boundary on an equivalent heart valve structure (e.g., mitral valve structures of different subjects). The outline defines at least one boundary of the target structure. Because the number of training sets of the training data 1025 may be of smaller size than data sets typically used for deep learning, transformations (e.g., rotations, stretching, scaling, etc.) may be applied to the training data to increase the size of the learning data. The training data 1025 may include synthetic data generated by scanning phantoms, or exams from other imaging modalities such as MRI translated to CT using GANs.

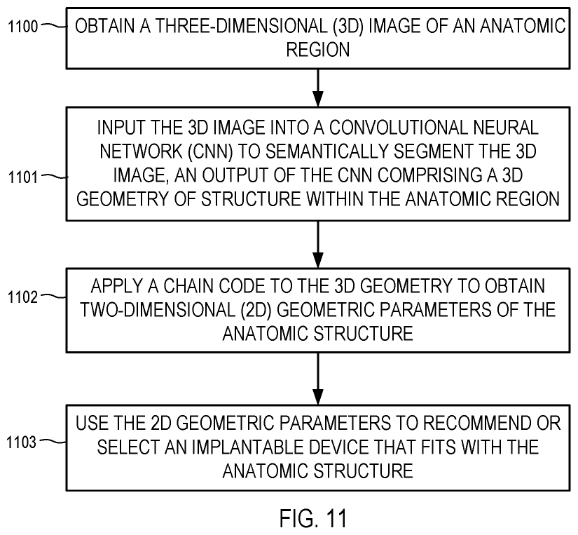

In FIG. 11, a flowchart shows a method according to an example embodiment, e.g., that may be implemented via a system 1000 as shown in FIG. 10. The method involves obtaining 1100 a 3D image of an anatomic region such as a cardiac region. The 3D image is input 1101 into a CNN to semantically segment the 3D image. The CNN produces an output that includes a 3D geometry of an anatomical structure (e.g., heart valve structure) within the anatomic region. A chain code is applied 1102 to the 3D geometry to obtain 2D geometric parameters of the anatomic structure. The 2D geometric parameters are used 1103 to recommend or select an implantable device that fits with the anatomic structure

The various embodiments described above may be implemented using circuitry, firmware, and/or software modules that interact to provide particular results. One having skill in the arts can readily implement such described functionality, either at a modular level or as a whole, using knowledge generally known in the art. For example, the flowcharts and control diagrams illustrated herein may be used to create computer-readable instructions/code for execution by a hardware processor. Such instructions may be stored on a non-transitory, computer-readable medium and transferred to the processor for execution as is known in the art. The structures and procedures shown above are only a representative example of embodiments that can be used to provide the functions described hereinabove.

Note that any components described herein using terms such as “processor,” “controller,” “logic circuit,” or the like may be implemented using a plurality of discrete units operating together. For example, a processer that performs a series of steps or operations may be construed as two or more processors operating cooperatively to perform the steps. Similarly, other processing hardware such as memory and input-output may perform the described functions with multiple discrete units operating cooperatively or being coordinated by another unit, e.g., central processor or processors.

Unless otherwise indicated, all numbers expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein. The use of numerical ranges by endpoints includes all numbers within that range (e.g., 1 to 5 includes 1, 1.5, 2, 2.75, 3, 3.80, 4, and 5) and any range within that range.

The terms “coupled” or “connected” refer to elements being attached to each other either directly (in direct contact with each other) or indirectly (having one or more elements between and attaching the two elements). Either term may be modified by “operatively” and “operably,” which may be used interchangeably, to describe that the coupling or connection is configured to allow the components to interact to carry out at least some functionality.

Terms related to orientation, such as “top,” “bottom,” “side,” and “end,” are used to describe relative positions of components (e.g., as arranged in the figures) and are not meant to limit the orientation of the embodiments contemplated. For example, an embodiment described as having a “top” and “bottom” also encompasses embodiments thereof rotated in various directions unless the content clearly dictates otherwise.

Reference to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout are not necessarily referring to the same embodiment of the disclosure. Furthermore, the particular features, configurations, compositions, or characteristics may be combined in any suitable manner in one or more embodiment.

References to a “combination” of different elements is also meant to include each element on its own unless otherwise indicated. For example, a combination of A, B, and C may include any one of A, B, or C alone, as well as A+B, A+C, A+B+C, etc. Further, where the elements of the combinations are actions (e.g., steps of a method), the listing of actions is not meant to imply a specific order that the actions may be taken in the combination unless otherwise indicated.

The foregoing description of the example embodiments has been presented for the purposes of illustration and description. It is not intended to be exhaustive or to limit the embodiments to the precise form disclosed. Many modifications and variations are possible in light of the above teaching. Any or all features of the disclosed embodiments can be applied individually or in any combination and are not meant to be limiting, but purely illustrative. It is intended that the scope of the invention be limited not with this detailed description, but rather determined by the claims appended hereto.

1. A computer-implemented method comprising:
obtaining a three-dimensional (3D) image of a cardiac region;
inputting the 3D image into a convolutional neural network (CNN) to semantically segment the 3D image, an output of the CNN comprising a 3D geometry of a heart valve structure within the cardiac region;
applying a chain code to the 3D geometry to obtain two-dimensional (2D) geometric parameters of the heart valve structure; and
using the 2D geometric parameters to recommend or select an implantable device that fits with the heart valve structure.

2. The method of claim 1, wherein the heart valve structure comprises a mitral valve annulus, and wherein the implantable device comprises a transcatheter mitral valve replacement system.

3. The method of claim 2, wherein the 2D geometric parameters include the following parameters of the mitral valve annulus: an inter-commissural diameter, an anterior-posterior diameter, a perimeter, and an area.

4. The method of claim 1, wherein inputting the 3D image into the CNN comprises:
inputting a 3D heart scan image into a first CNN that produces a bounding volume that contains the heart valve structure;
bounding the 3D heart scan image by the bounding volume; and
inputting the bounded image to a second CNN to obtain the 3D geometry of the heart valve structure.

5. : The method of claim 1, further comprising training the CNN on patient data sets in which a clinician manually defines a 2D outline on a 3D heart image, the 2D outline defining at least one boundary of an equivalent heart valve structure.

6. The method of claim 1, wherein the 3D geometry of the heart valve structure has a volume that is less than 0.1% of the 3D image.

7. The method of any claim 1, wherein applying the chain code to the 3D geometry comprises projecting the 3D geometry to a plane, the chain code being applied in 2D to the projection.

8. The method of claim 1, wherein the 3D image is constructed from a time series of scanned 3D images.

9. The method of claim 1, wherein the 3D image comprises a computed tomography (CT) image.

10. The method of claim 9, wherein the CNN is trained on any combination of CT images, synthetic data generated by scanning phantoms, and different modality images obtained from imaging modalities other than CT, the different modality images translated to a CT input using one or more generative adversarial networks (GANs).

11. A non-transitory computer-readable medium comprising instructions that, when executed by a processor, perform the method of claim 1.

12. A computer-implemented method comprising:
obtaining a three-dimensional (3D) image of a cardiac region;
inputting the 3D image into a convolutional neural network (CNN) to semantically segment the 3D image, an output of the CNN comprising a 3D geometry of a mitral valve annulus;
applying a chain code to the 3D geometry to obtain two-dimensional (2D) geometric parameters of the mitral valve annulus; and
using the 2D geometric parameters to recommend or select transcatheter mitral valve replacement that fits with the mitral valve annulus while minimizing risk of left ventricular outflow tract obstruction.

13. The method of claim 12, wherein the 2D geometric parameters include the following parameters of the mitral valve annulus: an inter-commissural diameter, an anterior-posterior diameter, a perimeter, and an area.

14. The method of claim 12, wherein inputting the 3D image into the CNN comprises:
inputting a 3D heart scan image into a first CNN that produces a bounding volume that contains the mitral valve annulus;
bounding the 3D heart scan image by the bounding volume; and
inputting the bounded image to a second CNN to obtain the 3D geometry of the mitral valve annulus.

15. The method of claim 12, further comprising training the CNN on patient data sets in which 2D outlines are superimposed over mitral annuli on 3D heart images.

16. The method of claim 12, wherein the 3D geometry of the mitral annulus has a volume that is less than 0.1% of the 3D image.

17. The method of claim 12, wherein applying the chain code to the 3D geometry comprises projecting the 3D geometry to a plane, the chain code being applied in 2D to the projection.

18. The method of claim 12, wherein the 3D image is constructed from a time series of scanned 3D images.

19. The method of claim 12, wherein the 3D image comprises a computed tomography (CT) image, and wherein the CNN is trained on any combination of CT images, synthetic data generated by scanning phantoms, and different modality images obtained from imaging modalities other than CT, the different modality images translated to a CT input using one or more generative adversarial networks (GANs).

20. A non-transitory computer-readable medium comprising instructions that, when executed by a processor, perform the method of claim 12.
